(19) United States
(12) Patent Application Publication
(10) Pub. No.: US 2024/0180961 A1
(43) Pub. Date: Jun. 6, 2024

(54) PLURIPOTENT STEM CELL-DERIVED HEMATOPOIETIC LINEAGES

(71) Applicant: GARUDA THERAPEUTICS, INC., Cambridge, MA (US)

(72) Inventor: Dhvanit SHAH, Chestnut Hill, MA (US)

(21) Appl. No.: 18/284,577

(22) PCT Filed: Mar. 30, 2022
(86) PCT No.: PCT/US22/22562

§ 371 Date: Sep. 28, 2023

Related U.S. Application Data
(60) Provisional application No. 63/168,360, filed on Mar. 31, 2021.

(51) Int. Cl. A61K 35/17; A61K 39/00; C12N 5/00; C12N 5/0783

(52) U.S. Cl. CPC A61K 35/17; A61K 39/4611; A61K 39/4613; A61K 39/4631; C12N 5/0018; C12N 5/0637; C12N 5/0646; A61K 2239/48; C12N 2501/25; C12N 2501/41; C12N 2501/42; C12N 2501/999; C12N 2506/45; C12N 2510/00; C12N 2513/00; C12N 2533/74

(57) ABSTRACT

The present disclosure, in various aspects and embodiments, provides methods for generating hematopoietic lineage cells for hematopoietic cell therapy. The various hematopoietic lineages include T lymphocytes including progenitor T cells, natural killer cells, B lymphocytes, neutrophils, monocytes and/or macrophages, red cells, megakaryocytes, and platelets. In various embodiments, the invention provides for efficient ex vivo processes for developing hematopoietic lineages, including but not limited to progenitor T cells and T cell lineages, from human induced pluripotent stem cells (iPSCs). Cells generated according to the disclosure in various embodiments are functional and/or more closely resemble the corresponding lineage isolated from peripheral blood or lymphoid organs. The present invention in some aspects provides isolated cells and cell compositions produced by the methods disclosed herein, as well as methods for adoptive cell therapy.

### CROSS-REFERENCE TO RELATED APPLICATIONS

This application claims the benefit of and priority to U.S. Provisional Patent Application No. 63/168,360, filed Mar. 31, 2021, the contents of which are hereby incorporated by reference in their entirety.

### BACKGROUND

The generation of hematopoietic cells from pluripotent cells ex vivo has attracted the interest of the scientific community for its prospects for allogeneic compatible cell-based therapies. Induced pluripotent stem cells (iPSCs) could potentially serve as a supply for generating “off-the-shelf” therapeutic lymphocytes. Nianias, A., & Themeli, M., Induced pluripotent stem cell (iPSC)-derived lymphocytes for adoptive cell immunotherapy: recent advances and challenges. Current Hematologic Malignancy Reports, 14(4), 261-268 (2019). However, methods for making clinically relevant numbers of hematopoietic cell lineages (e.g., immune cells, lymphocytes, etc.) and making cell lineages having clinically advantageous phenotypes, remain significant hurdles. In various aspects and embodiments, the invention meets these objectives.

### SUMMARY OF THIS DISCLOSURE

The present disclosure, in various aspects and embodiments, provides methods for generating hematopoietic lineages. The various hematopoietic lineages include T lymphocytes (T cells, including progenitor T cells), natural killer cells (NK cells), B lymphocytes (B cells) (including B-cells designed to generate specific antibodies), neutrophils, monocytes and/or macrophages, megakaryocytes, red cells, and platelets. Cells generated according to the disclosure in various embodiments are functional and/or more closely resemble the corresponding lineage isolated from peripheral blood or lymphoid organs. The present invention in some aspects provides isolated cells and cell compositions produced by the methods disclosed herein, as well as methods for cell therapy.

In one aspect, the disclosure provides a method for preparing a cell population of a hematopoietic lineage. The method comprises preparing a pluripotent stem cell (PSC) population, such as an induced pluripotent stem cell (iPSC) population differentiated to embryoid bodies, and enriching for CD34+ cells to thereby prepare a CD34-enriched population. Endothelial-to-hematopoietic transition (EHT) is induced in the CD34-enriched population to thereby prepare a hematopoietic stem cell (HSC) population, followed by differentiation to a hematopoietic lineage. In various embodiments, the hematopoietic lineage is selected from T lymphocytes (i.e., T cells), progenitor T cells, natural killer cells (NK cells), B lymphocytes (i.e., B cells), monocyte and/or macrophage, megakaryocytes, and platelets. In accordance with aspects and embodiments of this disclosure, it is discovered that inducing endothelial-to-hematopoietic transition (EHT) of a CD34+ cell population, which are derived from iPSCs-embryoid bodies, can be used for the ex vivo generation of superior hematopoietic lineages.

In some aspects and embodiments, the disclosure provides a method for generating a CD7+ progenitor T cell population, or a derivative of this population. For example, the method comprises generating a hematopoietic stem cell (HSC) population comprising human long-term hematopoietic stem cells (LT-HSCs) from iPSCs (e.g., hiPSCs). The HSC population is derived by induction of endothelial-to-hematopoietic transition of CD34+ cells (e.g., CD34+ cells derived from embryoid bodies). The HSC population (or cells isolated therefrom) is cultured with a partial or full Notch ligand (including but not limited to DLL4, DLL1, SFIP, etc.), sonic hedgehog (SHH), TNF-alpha, RetroNectin (or other extracellular matrix components), and/or combinations thereof, to produce a population comprising CD7+ progenitor T cells or a derivative cell population. The present disclosure provides HSC populations generated ex vivo from iPSCs and which respond to Notch ligand, sonic hedgehog (SHH), and/or component(s) of extracellular matrix, by robust production of T progenitor cells and T cell lineages ex vivo.

In various embodiments, the iPSCs are prepared by reprogramming somatic cells, such as but not limited to fibroblasts or PBMCs (or cells isolated therefrom). In some embodiments, the iPSCs are derived from lymphocytes, cord blood cells, PBMCs, CD34+ cells, or other human primary tissues. In some embodiments, iPSCs are derived from CD34+ cells isolated from peripheral blood. In various embodiments, the iPSCs can be gene edited to assist in HLA matching, such as by deletion of one or more HLA Class I and/or Class II alleles or their master regulators.

In some embodiments, hiPSCs are used to generate embryoid bodies (EB), which can be used for generation of (i.e., isolation or enrichment of) CD34+ cells. For example, EBs can be dissociated, and the CD34+ hematopoietic precursors isolated or enriched. In some embodiments, the process according to each aspect can comprise generating CD34-enriched cells from the pluripotent stem cells (e.g., EBs) and inducing endothelial-to-hematopoietic differentiation.

In some embodiments, CD34 enrichment and EHT may be induced at Day 8 to Day 14 of iPSC differentiation. EHT can be induced using any process. In some embodiments, induction of EHT generates a hematopoietic stem cell (HSC) population comprising LT-HSCs. In some embodiments, EHT generates HSCs through endothelial or hemogenic endothelial cell (HEC) precursors using mechanical, biochemical, pharmacological and/or genetic means. In some embodiments, the method comprises increasing the expression or activity of dnmt3b in PSCs, EBs, CD34-enriched cells, ECs, HECs or HSCs, which can be by mechanical, genetic, biochemical, or pharmacological means.

In some embodiments, cells are contacted with an effective amount of an agonist of a mechanosensitive receptor or a mechanosensitive channel that increases the activity or expression of Dnmt3b. In some embodiments, the mechanosensitive receptor is Piezol. Exemplary Piezol agonists include Yoda1, Jedi1, and Jedi2. In some embodiments, the mechanosensitive receptor is Trpv4. An exemplary Trpv4 agonist is GSK1016790A. In certain embodiments, Piezol activation is applied at least to CD34+ cells isolated from EBs, which in accordance with various embodiments, allows for superior generation of T progenitor cells as compared to other methods for inducing EHT.

In some embodiments, the method comprises applying cyclic 2D, 3D, or 4D stretch to cells. In various embodiments, the cells subjected to cyclic 2D, 3D, or 4D stretch are selected from one or more of CD34-enriched cells, iPSCs, ECs, and HECs. The cyclic-strain biomechanical stretching can increase the activity or expression of Dnmt3b and/or Gimap6.

Generally, at various steps, cell populations can be enriched for cells of a desired phenotype, and/or depleted of cells of an undesired phenotype. In some embodiments, cells are enriched for CD34+ cells (prior to and/or after undergoing EHT). In some embodiments, the cell population is cultured under conditions that promote expansion of CD34+ cells to thereby produce an expanded population of stem cells. Hematopoietic stem cells (HSCs) which give rise to erythroid, myeloid, and lymphoid lineages, can be identified based on the expression of CD34 and the absence of lineage specific markers (termed Lin−).

In various embodiments, the HSC population or fraction thereof (e.g., CD34+ fraction) is differentiated to a hematopoietic lineage, which can be selected from progenitor T cells, T cells and fractions thereof, B cells, NK cells, neutrophils, monocytes or macrophages, megakaryocytes, red cells, and platelets.

In some embodiments, the cell population is cultured with at least a Notch ligand, partial or full, (including but not limited to DLL4, DLL1, SFIP, etc.), sonic hedgehog (SHH), TNF-alpha, RetroNectin (or other extracellular matrix components), and/or combinations thereof, ex vivo to differentiate HSCs to CD7⁺ progenitor T cells, and optionally to a T cell lineage or other lineage (e.g., NK cell). In various embodiments, the Notch ligand comprises at least one of Delta-Like-1 (DL1) and Delta-Like-4 (DL4), SFIP, or a functional portion thereof. In various embodiments, the Notch ligand is immobilized, functionalized, and/or embedded in 2D or 3D culture system. The Notch ligand may be incorporated along with a component of extracellular matrix.

In other aspects, the invention provides a cell population, or pharmaceutically acceptable composition thereof, produced by the method described herein, as well as methods of treatment or use in therapy. In some embodiments, the cell population is a lymphocyte population (such as a T cell progenitor population) capable of engraftment in a thymus, spleen, or secondary lymphoid organ upon administration to a subject in need.

Various other aspects and embodiments of the disclosure will be apparent from the following detailed description.

### BRIEF DESCRIPTION OF DRAWINGS

FIG. 1 shows that ETV2 over-expression (OE) does not affect pluripotency. FIG. 1 shows FACS plots representative of transduction efficiency of iPSC with an adenoviral vector to overexpress ETV2 and GFP sequences. ETV2 overexpression does not affect the iPSC stemness as shown by the expression of the TRA-1-60 stemness marker.

FIG. 2 shows that ETV2 over-expression (OE) increases the yield of hemogenic endothelial cells. Representative flow cytometric analysis of hemogenic endothelial cells (described as CD235a−CD34+CD31+) and relative quantification demonstrates that ETV2-OE enhances the formation of hemogenic endothelial cells.

FIG. 3 shows that ETV2 over-expression (OE) enhances CD34+ cell formation during iPSC differentiation. Representative flow cytometric analysis of CD34+ cells and relative quantification demonstrates that ETV2-OE enhances the CD34+ cell formation.

FIG. 4A and FIG. 4B show that iPSC-derived HSCs that are derived with Piezo1 activation undergo pro-T cell differentiation similar to bone marrow (BM)-HSCs. FIG. 4A is a FACS plot of differentiation efficiency to CD34+CD7+ pro T cells of Bone Marrow (BM) HSCs and iPSC-HSCs derived with Piezol activation. FIG. 4B is a quantification of CD34+CD7+ cells (%) derived with (1) BM-HSCs and (2) iPSC-HSCs (Piezol Activation). FIG. 4B shows the average of three experiments.

FIG. 5A and FIG. 5B show that iPSC-derived HSCs generated with Piezo1 activation undergo T cell differentiation and can be activated with CD3/CD28 beads similar to BM-HSCs. FIG. 5A is a FACS plot of activation efficiency (CD3+CD69+ expression) of T cells differentiated from BM-HSCs and iPSC-derived HSCs generated with Piezo1 activation. FIG. 5B is a quantification of CD3+CD69+ cells (%) derived with (1) BM-HSCs and (2) iPSC-HSCs (Piezol Activation). FIG. 5B shows the average of three experiments.

FIG. 6 shows that iPSC-derived HSCs generated with Piezol activation can differentiate to functional T cells. IFNγ expression is a consequence of T cell activation after T cell receptor (TCR) stimulation via CD3/CD28 beads. IFNγ expression in T cells differentiated from iPSC-derived HSCs, generated upon Piezol activation, enhances HSC ability to further differentiate to functional T cells. FIG. 6 shows the average of three experiments.

### DESCRIPTION OF THE INVENTION

The present disclosure, in various aspects and embodiments, provides methods for generating hematopoietic lineages for cell therapy. The various hematopoietic lineages include T lymphocytes (T cells, including progenitor T cells), natural killer cells (NK cells), B lymphocytes (B cells) (including B-cells designed to generate specific antibodies), neutrophils, monocytes and/or macrophages, megakaryocytes, red cells, and platelets. In various embodiments, the invention provides for efficient ex vivo processes for developing hematopoietic lineages, including but not limited to progenitor T cells and T cell lineages, from human induced pluripotent stem cells (iPSCs). Cells generated according to the disclosure in various embodiments are functional and/or more closely resemble the corresponding lineage isolated from peripheral blood or lymphoid organs. The present invention in some aspects provides isolated cells and cell compositions produced by the methods disclosed herein, as well as methods for cell therapy.

In accordance with aspects and embodiments of this disclosure, the ability of human induced pluripotent stem cells (hiPSCs) to produce essentially limitless pluripotent stem cells (PSCs) is leveraged to generate boundless supply of hematopoietic cells, including but not limited to therapeutic human T lymphocytes (“T cells”) or their progenitors. Use of primary T cells as therapeutic lymphocytes has been limited by their restricted availability, cell numbers, limited expansion potential, and histocompatibility issues. Moreover, compared to primary cells, hiPSCs can more readily undergo genetic modifications in vitro, thereby offering opportunities to improve cell-target specificity, cell numbers, as well as bypassing HLA-matching issues for example. Additionally, fully engineered hiPSC clones, as compared to primary cells, can serve as a stable and safe source (Nianias and Themeli, 2019). Further, because hiPSCs, unlike human Embryonic Stem Cells (hESCs), are of non-embryonic origin, they are also free of ethical concerns. Accordingly, use of hiPSCs according to this disclosure confers several advantages over primary cells to generate therapeutic hematopoietic lineages, such as T lymphocytes.

In one aspect, the disclosure provides a method for preparing a cell population of a hematopoietic lineage. The method comprises preparing a pluripotent stem cell (PSC) population, such as an induced pluripotent stem cell (iPSC) population differentiated to embryoid bodies, and enriching for CD34+ cells to thereby prepare a CD34-enriched population. Endothelial-to-hematopoietic transition (EHT) is induced in the CD34-enriched population to thereby prepare a hematopoietic stem cell (HSC) population, optionally followed by a further enrichment of CD34+ cells. The resulting HSC population (or fraction thereof) is differentiated to a hematopoietic lineage. In various embodiments, the hematopoietic lineage is selected from T lymphocytes (i.e., T cells), progenitor T cells, natural killer cells (NK cells), B lymphocytes (i.e., B cells), monocyte and/or macrophage, megakaryocytes, and platelets.

Conventionally, hematopoietic lineages are prepared by differentiation of iPSCs to embryoid bodies up to day 8 to harvest CD34+ cells. CD34 is commonly used as a marker of hemogenic endothelial cells, hematopoietic stem cells, and hematopoietic progenitor cells. In accordance with aspects and embodiments of this disclosure, it is discovered that inducing endothelial-to-hematopoietic transition (EHT) of a CD34+ cell population, and which can be derived from iPSCs-embryoid bodies, can be used for the ex vivo generation of superior hematopoietic lineages.

In some aspects and embodiments, the disclosure provides a method for generating a CD7+ progenitor T cell population, or a derivative of this population. For example, the method comprises generating a hematopoietic stem cell (HSC) population comprising human long-term hematopoietic stem cells (LT-HSCs) from iPSCs (e.g., hiPSCs). The HSC population is derived by induction of endothelial-to-hematopoietic transition of CD34+ cells (e.g., CD34+ cells derived from embryoid bodies). The HSC population (or cells isolated therefrom) is cultured with a partial or full Notch ligand, sonic hedgehog (SHH), RetroNectin (or other extracellular matrix component(s)), and/or combinations thereof, to produce a population comprising CD7+ progenitor T cells or a derivative cell population.

The Notch signaling pathway regulates the formation, differentiation, and function of progenitor T-cells, pre-T cells, and/or mature T lymphocytes. In vivo, T cell development proceeds after lymphocyte progenitors differentiate from bone marrow hematopoietic stem cells and migrate to the thymus. Specialized thymic epithelial cells induce T cells to develop along a controlled pathway. Notch signaling plays a critical role during T lineage commitment in the thymus. As lymphoid progenitors enter the thymus, they encounter dense expression of Notch ligands on thymic epithelium that drives thymopoiesis. The present disclosure provides HSC populations generated ex vivo from iPSCs and which respond to Notch ligand, SHH, and/or component(s) of extracellular matrix, by robust production of T progenitor cells and T cell lineages ex vivo.

In various embodiments, the iPSCs are prepared by reprogramming somatic cells. The term “induced pluripotent stem cell” or “iPSC” refers to cells derived from somatic cells, such as skin or blood cells that have been reprogrammed back into an embryonic-like pluripotent state. In some embodiments, iPSCs are generated from somatic cells such as (but not limited to) fibroblasts or PBMCs (or cells isolated therefrom). In some embodiments, the iPSCs are derived from lymphocytes (e.g., T-cells, B-cells, NK-cells, etc.), cord blood cells (including from CD3+ or CD8+ cells from cord blood), PBMCs, CD34+ cells, or other human primary tissues. In some embodiments, iPSCs are derived from CD34+ cells isolated from peripheral blood. In various embodiments, the iPSCs are autologous or allogenic (e.g., HLA-matched at one or more loci) with respect to a recipient (a subject in need of treatment as described herein). In various embodiments, the iPSCs can be gene edited to assist in HLA matching (such as deletion of one or more HLA Class I and/or Class II alleles or their master regulators, including but not limited beta-2-microglobulin (β2M), CIITA, etc.), or gene edited to delete or express other functionalities. For example, iPSCs can be gene edited to delete one or more of HLA-A, HLA-B, and HLA-C, and to delete one or more of HLA-DP, HLA-DQ, and HLA-DR. In certain embodiments, the iPSCs retain expression of at least one HLA Class I and at least one HLA Class II complex. In certain embodiments, iPSCs are homozygous for at least one retained Class I and Class II loci. In some embodiments, iPSCs are derived from T cells, for example, with a known or unknown TCR specificity. In some embodiments, the T cells bear TCRs with specificity for tumor associated antigens.

Somatic cells may be reprogrammed by expression of reprogramming factors selected from Sox2, Oct3/4, c-Myc, Nanog, Lin28, and klf4. In some embodiments, the reprogramming factors are Sox2, Oct3/4, c-Myc, Nanog, Lin28, and klf4. In some embodiments, the reprogramming factors are Sox2, Oct3/4, c-Myc, and klf4. Methods for preparing iPSCs are described, for example, in U.S. Pat. Nos. 10,676,165; 9,580,689; and 9,376,664, which are hereby incorporated by reference in their entireties. In various embodiments, reprogramming factors are expressed using well known viral vector systems, such as lentiviral, Sendai, or measles viral systems. Alternatively, reprogramming factors can be expressed by introducing mRNA(s) encoding the reprogramming factors into the somatic cells. Further still, iPSCs may be created by introducing a non-integrating episomal plasmid expressing the reprogramming factors, i.e., for the creation of transgene-free and virus-free iPSCs. Known episomal plasmids can be employed with limited replication capabilities and which are therefore lost over several cell generations.

In some embodiments, the human pluripotent stem cells (e.g., iPSCs) are gene-edited. Gene-editing can include, but is not limited to, modification of HLA genes (e.g., deletion of one or more HLA Class I and/or Class II genes), deletion of β2 microglobulin (β2M), deletion of CIITA, deletion or addition of T Cell Receptor (TCR) genes, or addition of a chimeric antigen receptor (CAR) gene, for example. An exemplary CAR can target CD19, CD38, CD33, CD47, CD20, etc. For example, the iPSCs can be T-cell receptor (TCR)-transduced iPSCs. Such embodiments enable the production of large-scale regenerated T lymphocytes with a desired antigen-specificity. Alternatively, engineered iPSCs with one or more HLA knockouts and TCR knockouts can be placed in a bioreactor for a feeder-and-serum-free differentiation, under GMP-grade conditions, to generate fully functional and histocompatible T cells.

In some embodiments, iPSCs are prepared from CD3⁺ cells or in some embodiments T lymphocytes (e.g., CTLs) (T-iPSCs). For example, T lymphocytes can be isolated with a desired antigen specificity (using for example, cell sorting with HLA-peptide ligands), and reprogrammed to T-iPSCs. These T-iPSCS are then redifferentiated into progenitor T cells, or derivatives thereof or T cell lineages according to this disclosure. When T-iPSCs are produced from antigen-specific T cells, T-iPSCs inherit the rearranged T cell receptor (TCR) genes. In these embodiments, CTLs redifferentiated from the T-iPSCs demonstrate the same antigen specificity as the original CTLs.

In some embodiments, hiPSCs are used to generate embryoid bodies (EB), which can be used for generation of (i.e., isolation or enrichment of) CD34+ cells. For example, EBs can be dissociated, and the CD34+ hematopoietic precursors isolated or enriched. In some embodiments, human iPSC aggregates are expanded in a bioreactor as described, for example, in Abecasis B. et al., Expansion of 3D human induced pluripotent stem cell aggregates in bioreactors: Bioprocess intensification and scaling-up approaches. J. of Biotechnol. 246 (2017) 81-93.

In some embodiments, the process according to each aspect can comprise generating CD34-enriched cells from the pluripotent stem cells (e.g., EBs) and inducing endothelial-to-hematopoietic differentiation. HSCs comprising relatively high frequency of LT-HSCs can be generated from the cell populations using various stimuli or factors, including mechanical, biochemical, metabolic, and/or topographical stimuli, as well as factors such as extracellular matrix, niche factors, cell-extrinsic factors, induction of cell-intrinsic properties; and including pharmacological and/or genetic means.

In some embodiments, the method comprises preparing endothelial cells with hemogenic potential from pluripotent stem cells, prior to induction of EHT. In some embodiments, the combined over-expression of GATA2/ETV2, GATA2/TAL1, or ER71/GATA2/SCL can lead to the formation of endothelial cells with hemogenic potential from PSC sources. In some embodiments, the method comprises overexpression of E26 transformation-specific variant 2 (ETV2) transcription factor in the iPSCs. Following CD34+ enrichment, HSCs are then generated from the endothelial cells using mechanical, biochemical, pharmacological and/or genetic stimulation or modification. ETV2 can be expressed by introduction of an encoding non-integrating episomal plasmid, for constitutive or inducible expression of ETV2, and for production of transgene-free hemogenic ECs. In some embodiments, ETV2 is expressed from an mRNA introduced into the iPSCs. mRNA can be introduced using any available method, including electroporation or lipofection. Differentiation of cells expressing ETV2 can comprise addition of VEGF-A. See, Wang K, et al., Robust differentiation of human pluripotent stem cells into endothelial cells via temporal modulation of ETV2 with mRNA. Sci. Adv. Vol. 6 (2020). Cells generated in this manner may be used for producing CD34+ cells and inducing EHT according to embodiments of this disclosure.

In some embodiments, CD34 enrichment and EHT may be induced at Day 8 to Day 14 of iPSC differentiation, such as for example, Day 8, Day 9, Day 10, Day 11, Day 12, Day 13, or Day 14. Differentiation of iPSCs can be according to known techniques. In some embodiments, iPSC differentiation involves factors such as, but not limited to, combinations of bFGF, Y27632, BMP4, VEGF, SCF, EPO, TPO, IL-6, IL-11, and/or IGF-1. In some embodiments, hPSCs are differentiated using feeder-free, serum-free, and/or GMP-compatible materials. In some embodiments, hPSCs are co-cultured with murine bone marrow-derived feeder cells such as OP9 or MS5 cell line in serum-containing medium. The culture can contain growth factors and cytokines to support differentiation of embryoid bodies or monolayer system. The OP9 co-culture system can be used to generate multipotent HSPCs, which can be differentiated further to several hematopoietic lineages including T lymphocytes, B lymphocytes, megakaryocytes, monocytes or macrophages, and erythrocytes. See Netsrithong R. et al., Multilineage differentiation potential of hematoendothelial progenitors derived from human induced pluripotent stem cells, Stem Cell Research & Therapy Vol. 11 Art. 481 (2020). Alternatively, a step-wise process using defined conditions with specific signals can be used. For example, the expression of HOXA9, ERG, RORA, SOX4, and MYB in human PSCs favors the direct differentiation into CD34+/CD45+ progenitors with multilineage potential. Further, expression of factors such as HOXB4, CDX4, SCL/TAL1, or RUNX1a support the hematopoietic program in human PSCs. See Doulatov S. et al., Induction of multipotential hematopoietic progenitors from human pluripotent stem cells via re-specification of lineage-restricted precursors, Cell Stem Cell. 2013 Oct. 3; 13(4).

Induction of EHT can be with any known process. In some embodiments, induction of EHT generates a hematopoietic stem cell (HSC) population comprising LT-HSCs. In some embodiments, EHT generates HSCs through endothelial or hemogenic endothelial cell (HEC) precursors using mechanical, biochemical, pharmacological and/or genetic means (e.g., via stimulation, inhibition, and/or genetic modifications). In some embodiments, the EHT generates a stem cell population comprising one or more of long-term hematopoietic stem cells (LT-HSCs), short-term hematopoietic stem cells (ST-HSCs), and hematopoietic stem progenitor cells.

In some embodiments, the method comprises increasing the expression or activity of dnmt3b in PSCs, embryoid bodies, CD34-enriched cells, ECs, HECs or HSCs, which can be by mechanical, genetic, biochemical, or pharmacological means. In some embodiments, the method comprises increasing activity or expression of DNA (cytosine-5-)-methyltransferase 3 beta (Dnmt3b) and/or GTPase IMAP Family Member 6 (Gimap6) in the cells. See WO 2019/236943 and WO 2021/119061, which is hereby incorporated by reference in its entirety. In some embodiments, the induction of EHT comprises increasing the expression or activity of dnmt3b.

In some embodiments, cells are contacted with an effective amount of an agonist of a mechanosensitive receptor or a mechanosensitive channel that increases the activity or expression of Dnmt3b. In some embodiments, the mechanosensitive receptor is Piezol. An exemplary Piezol agonist is Yoda1. In some embodiments, the mechanosensitive receptor is Trpv4. An exemplary Trpv4 agonist is GSK1016790A. Yoda1 (2-[5-[[(2,6-Dichlorophenyl)methyl]thio]-1,3,4-thiadiazol-2-yl]-pyrazine) is a small molecule agonist developed for the mechanosensitive ion channel Piezol. Syeda R, Chemical activation of the mechanotransduction channel Piezol. eLife (2015). Yoda 1 has the following structure:

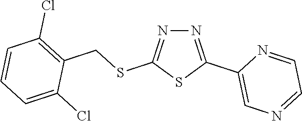 [SMILES: Clc1cccc(Cl)c1CSc1nnc(-c2cnccn2)s1]

Derivatives of Yoda1 can be employed in various embodiments. For example, derivatives comprising a 2,6-dichlorophenyl core are employed in some embodiments. Exemplary agonists are disclosed in Evans E L, et al., Yoda1 analogue (Dooku1) which antagonizes Yoda1-evoked activation of Piezol and aortic relaxation, British J. of Pharmacology 175(1744-1759): 2018. Still other Piezol agonist include Jedi1, Jedi2, and derivatives and analogues thereof. See Wang Y., et al., A lever-like transduction pathway for long-distance chemical- and mechano-gating of the mechanosensitive Piezol channel. Nature Communications (2018) 9:1300. These Piezol agonists are commercially available. In various embodiments, the effective amount of the Piezol agonist or derivative is in the range of about 1 μM to about 500 μM, or about 5 μM to about 200 μM, or about 5 μM to about 100 μM, or in some embodiments, in the range of about 25 μM to about 150 μM, or about 25 μM to about 100 μM, or about 25 μM to about 50 μM.

In various embodiments, pharmacological Piezol activation is applied to CD34+ cells (i.e., CD34-enriched cells). In certain embodiments, pharmacological Piezol activation may further be applied to iPSCs, embryoid bodies, ECs, hemogenic endothelial cells (HECs), HSCs, hematopoietic progenitors, as well as hematopoietic lineage(s). In certain embodiments, Piezol activation is applied at least to EBs generated from iPSCs, CD34+ cells isolated from EBs, and/or combinations thereof, which in accordance with various embodiments, allows for superior generation of T progenitor cells as compared to other methods for inducing EHT.

Alternatively or in addition, the activity or expression of Dnmt3b can be increased directly in the cells, e.g., in CD34-enriched cells. For example, mRNA expression of Dnmt3b can be increased by delivering Dnmt3b-encoding transcripts to the cells, or by introducing a Dnmt3b-encoding transgene, or a transgene-free method, not limited to introducing a non-integrating episome to the cells. In some embodiments, gene editing is employed to introduce a genetic modification to Dnmt3b expression elements in the cells, such as, but not limited to, to increase promoter strength, ribosome binding, RNA stability, and/or impact RNA splicing.

In some embodiments, the method comprises increasing the activity or expression of Gimap6 in the cells, alone or in combination with Dnmt3b and/or other genes that are up- or down regulated upon cyclic strain or Piezol activation. To increase activity or expression of Gimap6, Gimap6-encoding mRNA transcripts can be introduced to the cells, transgene-free approaches can also be employed, including but not limited, to introducing an episome to the cells; or alternatively a Gimap6-encoding transgene. In some embodiments, gene editing is employed to introduce a genetic modification to Gimap6 expression elements in the cells (such as one or more modifications to increase promoter strength, ribosome binding, RNA stability, or to impact RNA splicing).

In embodiments of this disclosure employing mRNA delivery to cells, known chemical modifications can be used to avoid the innate-immune response in the cells. For example, synthetic RNA comprising only canonical nucleotides can bind to pattern recognition receptors, and can trigger a potent immune response in cells. This response can result in translation block, the secretion of inflammatory cytokines, and cell death. RNA comprising certain non-canonical nucleotides can evade detection by the innate immune system, and can be translated at high efficiency into protein. See U.S. Pat. No. 9,181,319, which is hereby incorporated by reference, particularly with regard to nucleotide modification to avoid an innate immune response.

In some embodiments, expression of Dnmt3b and/or Gimap6 is increased by introducing a transgene into the cells, which can direct a desired level of overexpression (with various promoter strengths or other selection of expression control elements). Transgenes can be introduced using various viral vectors or transfection reagents (including Lipid Nanoparticles) as are known in the art. In some embodiments, expression of Dnmt3b and/or Gimap6 is increased by a transgene-free method (e.g., episome delivery). In some embodiments, expression or activity of Dnmt3b and/or Gimap6 or other genes disclosed herein are increased using a gene editing technology, for example, to introduce one or more modifications to increase promoter strength, ribosome binding, or RNA stability.

Various editing technologies are known, which can be applied according to various embodiments of this disclosure. Gene editing technologies include but are not limited to CRISPR-Cas (e.g., CRISPR-Cas9), zinc fingers (ZFs), and transcription activator-like effectors (TALEs), etc. Fusion proteins containing one or more of these DNA-binding domains and the cleavage domain of Fokl endonuclease can be used to create a double-strand break in a desired region of DNA in a cell (See, e.g., US Patent Appl. Pub. No. US 2012/0064620, US Patent Appl. Pub. No. US 2011/0239315, U.S. Pat. No. 8,470,973, US Patent Appl. Pub. No. US 2013/0217119, U.S. Pat. No. 8,420,782, US Patent Appl. Pub. No. US 2011/0301073, US Patent Appl. Pub. No. US 2011/0145940, U.S. Pat. Nos. 8,450,471, 8,440,431, 8,440,432, and US Patent Appl. Pub. No. 2013/0122581, the contents of all of which are hereby incorporated by reference). In some embodiments, gene editing is conducting using CRISPR associated Cas system (e.g., CRISPR-Cas9), as known in the art. See, for example, U.S. Pat. Nos. 8,697,359, 8,906,616, and 8,999,641, which is hereby incorporated by reference in its entirety.

In some embodiments, the method comprises applying cyclic 2D, 3D, or 4D stretch to cells. In various embodiments, the cells subjected to cyclic 2D, 3D, or 4D stretch are selected from one or more of CD34-enriched cells, iPSCs, ECs, and HECs. For example, a cell population is introduced to a bioreactor that provides a cyclic-strain biomechanical stretching, as described in WO 2017/096215, which is hereby incorporated by reference in its entirety. The cyclic-strain biomechanical stretching can increase the activity or expression of Dnmt3b and/or Gimap6. In these embodiments, mechanical means apply stretching forces to the cells, or to a cell culture surface having the cells (e.g., ECs or HECs) cultured thereon. For example, a computer controlled vacuum pump system or other means for providing a stretching force (e.g., the FlexCell™ Tension System, the Cytostretcher System) attached to flexible biocompatible and/or biomimetic surface can be used to apply cyclic 2D, 3D, or 4D stretch ex vivo to cells under defined and controlled cyclic strain conditions. For example, the applied cyclic stretch can be from about 1% to about 20% cyclic strain (e.g., about 6% cyclic strain) for several hours or days (e.g., about 7 days). In various embodiments, cyclic strain is applied for at least about one hour, at least about two hours, at least about six hours, at least about eight hours, at least about 12 hours, at least about 24 hours, at least about 48 hrs, at least about 72 hrs, at least about 96 hrs, at least about 120 hrs, at least about 144 hrs, or at least about 168 hrs.

Alternatively or in addition, EHT is stimulated by Trpv4 activation. The Trpv4 activation can be by contacting cells (e.g., CD34-enriched cells, ECs, or HECs) with one or more Trpv4 agonists, which are optionally selected from GSK1016790A, 4alpha-PDD, or analogues and/or derivatives thereof.

Generally, at various steps, cell populations can be enriched for cells of a desired phenotype, and/or depleted of cells of an undesired phenotype. Such positive and negative selection methods are known in the art. For example, cells can be sorted based on cell surface antigens (including those described herein) using a fluorescence activated cell sorter, or magnetic beads which bind cells with certain cell surface antigens. Negative selection columns can be used to remove cells expressing undesired cell-surface markers. In some embodiments, cells are enriched for CD34+ cells (prior to and/or after undergoing EHT). In some embodiments, the cell population is cultured under conditions that promote expansion of CD34+ cells to thereby produce an expanded population of stem cells.

In various embodiments, CD34+ cells (e.g., the floater and/or adherent cells) are harvested from the culture undergoing endothelial-to-hematopoietic transition between Day 8 to Day 15 of iPSC differentiation.

In various embodiments, the HSCs or CD34-enriched cells are further expanded. For example, the HSCs or CD34-enriched cells can be expanded according to methods disclosed in U.S. Pat. Nos. 8,168,428; 9,028,811; 10,272,110; and 10,278,990, which are hereby incorporated by reference in their entireties. In some embodiments, ex vivo expansion of HSCs or CD34-enriched cells employs prostaglandin E₂ (PGE2) or a PGE₂ derivative. In some embodiments of this disclosure, the HSCs comprise at least about 0.01% LT-HSCs, or at least about 0.05% LT-HSCs, or at least about 0.1% LT-HSCs, or at least about 0.5% LT-HSCs, or at least about 1% LT-HSCs.

Hematopoietic stem cells (HSCs) which give rise to erythroid, myeloid, and lymphoid lineages, can be identified based on the expression of CD34 and the absence of lineage specific markers (termed Lin−). In some embodiments, a population of stem cells comprising HSCs are enriched, for example, as described in U.S. Pat. No. 9,834,754, which is hereby incorporated by reference in its entirety. For example, this process can comprise sorting a cell population based on expression of one or more of CD34, CD90, CD38, and CD43. A fraction can be selected for further differentiation that is one or more of CD34⁺, CD90⁺, CD38⁻, and CD43⁻. In some embodiments, the stem cell population for differentiation to a hematopoietic lineage is at least about 80% CD34⁺, or at least about 90% CD34⁺, or at least about 95% CD34⁺.

In some embodiments, the stem cell population, or CD34-enriched cells or fraction thereof, or derivative population are expanded as described in US 2020/0308540, which is hereby incorporated by reference in its entirety. For example, the cells are expanded by exposing the cells to an aryl hydrocarbon receptor antagonist including, for example, SR1 or an SR1-derivative. See also, Wagner et al., Cell Stem Cell 2016; 18(1): 144-55 and Boitano A., et al., Aryl Hydrocarbon Receptor Antagonists Promote the Expansion of Human Hematopoietic Stem Cells. Science 2010 Sep. 10; 329(5997): 1345-1348.

In some embodiments, the compound that promotes expansion of CD34⁺ cells includes a pyrimidoindole derivative including, for example, UM171 or UM729 (see US 2020/0308540, which is hereby incorporated by reference).

In some embodiments, the stem cell population or CD34-enriched cells are further enriched for cells that express Periostin and/or Platelet Derived Growth Factor Receptor Alpha (pdgfra) or are modified to express Periostin and/or pdgfra, as described in WO 2020/205969 (which is hereby incorporated by reference in its entirety). Such expression can be by delivering encoding transcripts to the cells, or by introducing an encoding transgene, or a transgene-free method, not limited to introducing a non-integrating episome to the cells. In some embodiments, gene editing is employed to introduce a genetic modification to expression elements in the cells, such as to modify promoter activity or strength, ribosome binding, RNA stability, or impact RNA splicing.

In still other embodiments, the stem cell population or CD34-enriched cells are cultured with an inhibitor of histone methyltransferase EZH1. Alternatively, EZH1 is partially or completely deleted or inactivated or is transiently silenced in the stem cell population. Inhibition of EZH1 can direct myeloid progenitor cells (e.g., CD34+CD45+) to lymphoid lineages. See WO 2018/048828, which is hereby incorporated by reference in its entirety. In still other embodiments, EZH1 is overexpressed in the stem cell population.

In various embodiments, the HSC population or fraction thereof is differentiated to a hematopoietic lineage, which can be selected from progenitor T cells, T cells and fractions thereof, B cells, B-cells custom designed to produce certain antibodies, NK cells, neutrophils, monocytes or macrophages, megakaryocytes, red cells, and platelets.

In some embodiments, the cell population is cultured with a Notch ligand, partial or full, SHH, extracellular matrix component(s), and/or combinations thereof, ex vivo, to differentiate HSCs to CD7⁺ progenitor T cells, and optionally to a T cell lineage or other lineage (e.g., NK cell). Further, according to known processes, xenogenic OP9-DL1 cells are often employed for differentiation to T cells. The OP9-DL1 co-culture system uses a bone marrow stromal cell line (OP9) transduced with the Notch ligand delta-like-1 (DLL1) to support T cell development from stem cell sources. The OP9-DL1 system limits the potential of the cells for clinical application. There is a need for feeder-cell-free systems that can generate T lymphocytes from hiPSCs for clinical use, and in some embodiments the present invention meets this objective.

The term “Notch ligand” as used herein refers to a ligand capable of binding to a Notch receptor polypeptide present in the membrane of a hematopoietic stem cell or progenitor T cell. The Notch receptors include Notch-1, Notch-2, Notch-3, and Notch-4. Notch ligands typically have a DSL domain (D-Delta, S-Serrate, and L-Lag2) comprising 20-22 amino acids at the amino terminus, and from 3 to 8 EGF repeats on the extracellular surface. In various embodiments, the Notch ligand comprises at least one of Delta-Like-1 (DLL1), Delta-Like-4 (DLL4), SFIP3, or a functional portion thereof. A key signal that is delivered to incoming lymphocyte progenitors by the thymus stromal cells in vivo is mediated by DL4, which is expressed by cortical thymic epithelial cells.

The earliest intrathymic progenitors express high levels of CD34 and CD7, do not express CD1a, and are triple-negative (TN) for mature T cell markers: CD4, CD8, and CD3. Commitment to the T cell lineage is associated with the expression of CD1a by CD7-expressing pro-thymocytes. Thus, immature stages of T-cell development are typically delineated as CD34⁺CD1a⁻ (most immature) and CD34⁺CD1a⁻ cells. The transition from CD34⁺CD7⁺CD1a⁻ to CD34⁺CD7⁺CD1a⁺ by early thymocytes is associated with T-cell commitment. CD34⁺CD7⁺CD1a⁺ cells are likely T-lineage restricted. Following this stage, thymocytes progress to a CD4 immature single positive stage, at which point CD4 is expressed in the absence of CD8. Thereafter, a subset of the cells differentiates to the CD4⁺CD8⁺ double positive (DP) stage. Finally, following TCRα rearrangement, TCRαβ-expressing DP thymocytes undergo positive and negative selection, and yield CD4⁺CD8⁺ and CD4⁻CD8⁺ single positive (SP) T-cells.

In some embodiments, progenitor T cells are isolated by enrichment for CD7 expression. In some embodiments, progenitor T cells are expanded as described in US 2020/0308540, which is hereby incorporated by reference in its entirety. For example, the cells may be expanded by exposing the cells to an aryl hydrocarbon receptor antagonist including, for example, SR1 or an SR1-derivative. See also, Wagner et al., Cell Stem Cell 2016; 18(1): 144-55. In some embodiments, the compound that promotes expansion includes a pyrimidoindole derivative including, for example, UM171 or UM729 (see US 2020/0308540, which is hereby incorporated by reference).

Differentiation to progenitor T cells can further include in some embodiments the presence of stem cell factor (SCF), Flt3L and interleukin (IL)-7. In various embodiments, CD7+ progenitor T cells created express CD1a. The CD7+ progenitor T cells do not express CD34 or express a diminished level of CD34 compared to the HSC population. In some embodiments, the CD7+ progenitor T cells (or a portion thereof) further express CD5. Accordingly, the phenotype of the progenitor T cells may be CD7⁺CD1a⁺. In some embodiments, the phenotype of the progenitor T cells is CD7⁺CD5⁺. In some embodiments, the progenitor T cells are CD7⁺CD1a⁺CD5⁺, and optionally CD34⁺.

In some embodiments, the progenitor T cells exhibit a diminished level of CD34 expression, minimal CD34 expression (compared to the HSC population), or no CD34 expression. In some embodiments, CD34 expression is diminished in the population by at least about 50%, or at least about 75%, relative to the HSC population.

In some embodiments, the Notch ligand is an anti-Notch (agonistic) antibody that can bind and engage Notch signaling. In some embodiments, the antibody is a monoclonal antibody (including a human or humanized antibody), a single chain antibody (scFv), a nanobody, or other antibody fragment or antigen-binding molecule capable of activating the Notch signaling pathway.

In some embodiments, the Notch ligand is a Delta family Notch ligand. The Delta family ligand in some embodiments is Delta-1 (Genbank Accession No. AF003522, Homo sapiens), Delta-like 1 (DLL1, Genbank Accession No. NM_005618 and NP_005609, Homo sapiens; Genbank Accession No. X80903, 148324, M. musculus), Delta-4 (Genbank Accession No. AF273454, BAB18580, Mus musculus; Genbank Accession No. AF279305, AAF81912, Homo sapiens), and/or Delta-like 4 (DLL4; Genbank Accession. No. Q9NR61, AAF76427, AF253468, NM_019074, Homo sapiens; Genbank Accession No. NM 019454, Mus musculus). Notch ligands are commercially available or can be produced, for example, by recombinant DNA techniques.

In some embodiments, the Notch ligand comprises an amino acid sequence that is at least about 70%, or at least about 80%, or at least about 90%, or at least about 95%, or at least about 97% identical (e.g., about 100% identical) to human DLL1 or DLL4 Notch ligand. Functional derivatives of Notch ligands (including fragments or portions thereof) will be capable of binding to and activating a Notch receptor. Binding to a Notch receptor may be determined by a variety of methods known in the art including in vitro binding assays and receptor activation/cell signaling assays.

In various embodiments, the Notch ligands are soluble, and are optionally immobilized on microparticles or nanoparticles, which are optionally paramagnetic to allow for magnetic enrichment or concentration processes. In still other embodiments, the Notch ligands are immobilized on a 2D or 3D culture surface, optionally with other adhesion molecules such as VCAM-1. See US 2020/0399599, which is hereby incorporated by reference in its entirety. In other embodiments, the beads or particles are polymeric (e.g., polystyrene or PLGA), gold, iron dextran, or constructed of biological materials, such as particles formed from lipids and/or proteins. In various embodiments, the particle has a diameter or largest dimension of from about 0.01 μm (10 nm) to about 500 μm (e.g., from about 1 μm to about 7 μm). In still other embodiments, polymeric scaffolds with conjugated ligands can be employed, as described in WO 2020/131582, which is hereby incorporated by reference in its entirety. For example, scaffold can be constructed of polylactic acid, polyglycolic acid, PLGA, alginate or an alginate derivative, gelatin, collagen, agarose, hyaluronic acid, poly(lysine), polyhydroxybutyrate, poly-epsilon-caprolactone, polyphosphazines, poly(vinyl alcohol), poly(alkylene oxide), poly(ethylene oxide), poly(allylamine), poly(acrylate), poly(4-aminomethylstyrene), pluronic polyol, polyoxamer, poly(uronic acid), poly(anhydride), poly(vinylpyrrolidone), and any combination thereof. In some embodiments, the scaffold comprises pores having a diameter between about 1 μm and 100 μm.

In some embodiments, the C-terminus of the Notch ligand is conjugated to the selected support. In some embodiments, this can include adding a sequence at the C-terminal end of the Notch ligand that can be enzymatically conjugated to the support, for example, through a biotin molecule. In another embodiment, a Notch ligand-Fc fusion is prepared, such that the Fc segment can be immobilized by binding to protein A or protein G that is conjugated to the support. Of course, any of the known protein conjugation methods can be employed.

Thus, in various embodiments, the Notch ligand is immobilized, functionalized, and/or embedded in 2D or 3D culture system. The Notch ligand may be incorporated along with a component of extracellular matrix, such as one or more selected from fibronectin, RetroNectin, and laminin. In some embodiments, the Notch ligand and/or component of extracellular matrix are embedded in inert materials providing 3D culture conditions. Exemplary materials include, but are not limited to, cellulose, alginate, and combinations thereof. In some embodiments, the Notch ligand, a component of extracellular matrix, or combinations thereof, are in contact with culture conditions providing topographical patterns and/or textures (e.g., roughness) to cells conducive to differentiation and/or expansion.

In some embodiments, HSCs are differentiated to progenitor T cells by culture in medium comprising TNF-α and/or antagonist of aryl hydrocarbon/dioxin receptor (SR1), and in the presence of Notch ligand. See US 2020/0390817, US 2021/0169934, and US 2021/0169935, which are hereby incorporated by reference in its entirety. In some embodiments the HSCs are cultured in a medium comprising TNF-α, IL-7, thrombopoietin (TPO), Flt3L, and stem cell factor (SCF), and optionally SR1, in the presence of an immobilized Delta-Like-4 ligand and a fibronectin fragment. In some embodiments, the cells are cultured with RetroNectin, which is a recombinant human fibronectin containing three functional domains: the human fibronectin cell-binding domain (C-domain), heparin-binding domain (H-domain), and CS-1 sequence domain. In some embodiments, cells are cultured in the presence of an immobilized Delta-Like-4 ligand and a RetroNectin. In some embodiments, cells are cultured in the presence of an immobilized Delta-Like-4 ligand, TNF-alpha, and a RetroNectin. In some embodiments, cells are cultured in the presence of an immobilized Delta-Like-1 ligand and a RetroNectin. In some embodiments, cells are cultured in the presence of SFIP3 and RetroNectin. In some embodiments, cells are cultured in the presence of an immobilized Delta-Like-4 ligand and SHH molecules and/or functional derivatives thereof. Exemplary fibronectin fragments include one or more RGDS, CS-1, and heparin-binding motifs. Fibronectin fragments can be free in solution or immobilized to the culture surface or on particles. In some embodiments, cells are cultured for 5 to 7 days to prepare CD7+ progenitor T cells.

In various embodiments, the method produces progenitor T cells, or a T cell lineage, by culturing the HSC population with the Notch ligand (including any of the embodiments described above) with or without component(s) extracellular matrix, and optionally adding TNF-alpha to the culture at certain stages of differentiation. Thus, cells created in some embodiments are progenitor or precursor cells committed to the T cell lineage (“progenitor T cells”). In some embodiments, the cells are CD7⁺ progenitor T cells. In some embodiments, the cells are CD25⁺ immature T cells, or cells that have undergone CD4 or CD8 lineage commitment. In some embodiments, the cells are CD4⁺CD8⁺ double positive (DP), CD4⁻CD8⁺, or CD4⁺CD8⁻. In some embodiments, the cells are single positive (SP) cells that are CD4⁻CD8⁺ or CD4⁺CD8⁻ and TCR^{hi}. In some embodiments, the cells are TCRαβ⁺ and/or TCRγΔ⁺. In various embodiments, the cells are CD3⁺.

The adoptive transfer of progenitor T cells is a strategy for enhancing T cell reconstitution. Progenitor T cells are developmentally immature and undergo positive and negative selection in the host thymus. Thus, they become restricted to the recipient's major histocompatibility complex (MHC) yielding host tolerant T cells that can bypass the clinical challenges associated with graft-versus-host disease (GVHD). Importantly, engraftment with progenitor T cells restores the thymic architecture and improves subsequent thymic seeding by HSC-derived progenitors. In addition to its intrinsic regenerative medicine properties, progenitor T cells can also be engineered with T cell receptors (TCRs) and chimeric antigen receptors (CARs) (via either gene or mRNA delivery) to confer specificity to tumor-associated antigens.

In various embodiments, the progenitor T cells are further cultured under suitable conditions to generate cells of a desired T cell lineage, including with one or more Notch ligands. For example, the cells can be cultured in the presence of one or more Notch ligands as described for a sufficient time to form cells of the T cell lineage. In some embodiments, stem cells or progenitor T cells are cultured in suspension with soluble Notch ligand or Notch ligand conjugated to particles or other supports, or Notch ligand expressing cells. In some embodiments, the progenitor T cells or stem cells are cultured in suspension or in adherent format in a bioreactor, optionally a closed or a closed, automated bioreactor, with a soluble or conjugated Notch ligand in suspension. One or more cytokines, extracellular matrix component(s), and thymic niche factor(s) that promote commitment and differentiation to the desired T cell lineage may also be added to the culture or reactor. Such cytokines or factors are known in the art. In various embodiments, the HSC population is cultured with the Notch ligand for about 4 to about 21 days, or from about 6 to about 18 days, or from about 7 to about 14 days to generate progenitor T cells. In some embodiments, the stem cell population or derivative thereof is cultured for at least about 21 days or at least about 28 days to generate mature T cell lineages or NK cells.

In various embodiments, the HSC population is cultured in an artificial thymic organoid (ATO). See, Hagen, M. et al. (2019). The ATO will include culture of HSCs (or aggregates of HSCs) with a Notch ligand-expressing stromal cell line in serum-free conditions. The artificial thymic organoid is a 3D system, inducing differentiation of hematopoietic precursors to naive CD3⁺CD8⁺ and CD3⁺CD4⁺ T cells.

In various embodiments, the method comprises generating a derivative of the progenitor T cells or generating a T cell lineage from the progenitor T cells. In certain embodiments, the derivative of the progenitor T cell or T cell lineage expresses CD3 and a T cell receptor. In some embodiments, the T cell lineage is CD8⁺ and/or CD4⁺. For example, T cells lineages can include one or more of CD8⁺CD4⁻, CD8⁻CD4⁺, CD8⁺CD4⁺, and CD8⁻CD4⁻ cells. In some embodiments, the iPSCs, CD34+ cells, or derivatives thereof are modified to express a chimeric antigen receptor (CAR) at progenitor-T, T-cell, and/or NK cell level.

In some embodiments, the T cell lineage is a regulatory T cell. T regulatory cells (or T regs) are defined as CD4⁺CD25⁺. Tregs control the immune response to self and foreign antigens and help prevent autoimmune disease. Differentiation of progenitor T cells to Tregs in some embodiments involves culturing the progenitor T cells or Treg precursors with TGFβ and optionally IL-2 and/or IL-10.

In some embodiments, the HSC population or fraction thereof are differentiated to B lymphocytes (“B cells”). For example, culturing CD34+ or CD34+CD43+ cells with MS5 stromal cells or S17 stromal cells (e.g., for 15-25 days, or about 21 days) can generate a B-lymphoid identity with expression of CD19, CD45, and CD10. See Carpenter L. et al., Human induced pluripotent stem cells are capable of B-cell lymphopoiesis, Blood 117(15):4008-4011. Dubois F. et al., Toward a better definition of hematopoietic progenitors suitable for B cell differentiation, Plos One Dec. 15, 2020. In various embodiments, the B cells produced according to this disclosure express surface IgM (sIgM) and undergo VDJ rearrangement. In various embodiments, B cells produced according to this disclosure will engraft in the spleen and secondary lymphoid tissues of a subject for maturation.

In some embodiments, the HSC population or fraction thereof are differentiated to monocytes, macrophages, or neutrophils. For example, erythromyeloid precursors (EMP) (CD43+CD45+) may be generated by culture with IL-6, IL-3, thyroid peroxidase (TPO), SCF, FGF2, and VEGF, followed by differentiation to monocytes. Differentiation to monocytes to employ culture with M-CSF, IL-3, and IL-6. See Cao X et al., Differentiation and Functional Comparison of Monocytes and Macrophages from hiPSCs with Peripheral Blood Derivatives, Stem Cell Reports. 2019 Jun. 11; 12(6): 1282-1297. Monocytes and macrophage lineages prepared according to this disclosure are CD14+ and will exhibit endocytosis and phagocytic functions. In some embodiments, macrophages are polarized ex vivo to the M1 (pro-inflammatory) or M2 (immunosuppressive) phenotype. In some embodiments, CD45+ hematopoietic cells with phagocytic markers, such as CD33 and CD11b, are generated, and optionally subsequently to cells with neutrophil specific markers, such as CD66b, CD16b, GPI-80, etc., by differentiation of iPSC derived hCD34+ cells. These processes can employ differentiation media containing mixtures of cytokines and growth factors, including but not limited to SCF, IL3, FLT3, IL6, GM-CSF, G-CSF, EPO, TPO, and/or combinations thereof. In some embodiments, neutrophils and their precursors are generated by methods described in: Saeki L., et al., A Feeder-Free and Efficient Production of Functional Neutrophils from Human Embryonic Stem Cells, Stem Cells Vol. 27, Issue 1, 2009, Pages 59-67; Morishima T. et al., Neutrophil differentiation from human-induced pluripotent stem cells. J. Cell. Physiol. 226: 1283-1291, 2011; Yokoyama Y. et al., Derivation of functional mature neutrophils from human embryonic stem cells. Blood 2009 Jun. 25; 113(26):6584-92; and Sweeney C L et al., Generation of functionally mature neutrophils from induced pluripotent stem cells. Methods Mol Biol 2014; 1124:189-206.

In some embodiments, the HSC population or fraction thereof are differentiated to megakaryocytes or platelets. For example, megakaryocytes (as a renewable source for platelets) can be prepared from the HSCs or fraction thereof by culture with SCF, IL-11, and TPO for several days (e.g., about 5 days). Alternatively, other cytokines and growth factors such as IL-3, IL-6, SDF-1, and FGF-4 can be employed. Megakaryocytes will be CD42b+CD61+. See Liu L., Efficient Generation of Megakaryocytes From Human Induced Pluripotent Stem Cells Using Food and Drug Administration-Approved Pharmacological Reagents, Stem Cells Transl Med. 2015 April; 4(4): 309-319. Platelets can be further generated from megakaryocytes by culture in serum free media with IL-11. CD41+CD42a+ platelet-like-particles are recovered from the media.

In some embodiments, the derivative of the progenitor T cell is a natural killer (NK) cell. In some embodiments, NK cells are generated from progenitor T cells as described in U.S. Pat. No. 10,266,805, which is hereby incorporated by reference in its entirety. For example, the progenitor T cells can give rise to NK cells when cultured with IL-15. In some embodiments, the NK cell expresses a CAR, based on gene editing of iPSC, embryonic bodies, hCD34+ cells, or NK cells, or via mRNA expression in NK cells.

In some embodiments, the HSC population or fraction thereof is differentiated to red cells or derivatives thereof. Red cells produced according to this disclosure can be administered or used in therapy, for example, for an inherited or acquired red cell disorder, bone marrow failure disorder, high-altitude-related physiological and pathological condition, conditions related to chemicals or radiation exposure, and/or for treatment of subjects undergoing HSC transplant. In further embodiments, the red cells prepared according to this disclosure are provided as a pharmaceutical acceptable composition delivering or encapsulating drugs (including but not limited to enzymes), oxygen carriers, or other suitable materials to treat human disease or physiological or pathological conditions.

In other aspects, the invention provides a cell population, or pharmaceutically acceptable composition thereof, produced by the method described herein. In some embodiments, the cell population is a lymphocyte population capable of engraftment in a thymus, spleen, or secondary lymphoid organ upon administration to a subject in need. In various embodiments, the composition for cellular therapy is prepared that comprises the desired cell population a pharmaceutically acceptable vehicle. The pharmaceutical composition may comprise at least about 10² cells, or at least about 10³, or at least about 10⁴, or at least about 10⁵, or at least about 10⁶, or at least about 10⁷, or at least about 10⁸ cells. For example, in some embodiments, the pharmaceutical composition is administered, comprising from about 100,000 to about 400,000 cells per kilogram (e.g., about 200,000 cells/kg) of a recipient's body weight.

The cell composition of this disclosure may further comprise a pharmaceutically acceptable carrier or vehicle suitable for intravenous infusion or other administration route, and the composition may include a suitable cryoprotectant. An exemplary carrier is DMSO (e.g., about 10% DMSO). Cell compositions may be provided in unit vials or bags and stored frozen until use. In certain embodiments, the volume of the composition is from about one fluid ounce to one pint.

In some embodiments, this disclosure provides a CD7+ progenitor T cell, or pharmaceutically acceptable composition thereof, where the CD7+ progenitor T cell produced by a method disclosed herein. In various embodiments, the progenitor T cell is capable of engraftment in a thymus or spleen of a recipient. Progenitor T cells have the potential to decrease the risk of relapse of leukemia or other types of cancer in bone marrow transplant patients and to decrease the number of infections post-transplant that cause significant morbidity and mortality in patients. In another aspect, this disclosure provides a derivative of the progenitor T cell or T cell lineage produced by a method disclosed herein, or a pharmaceutically acceptable composition thereof.

In some embodiments, the cell population is a T cell population (or progenitor T cell population) or NK cell population, which are useful for adoptive cell therapy, for example, for human subjects having a condition selected from lymphopenia, a cancer, an immune deficiency, a viral infection, an autoimmune disease (particularly where the T cell population comprises Tregs), a skeletal dysplasia, a bone marrow failure syndrome, or a genetic disorder that impairs T cell development or function. Exemplary genetic disorders can impact the immune system, manifesting as an immunocompromised state, or autoimmune or pro-inflammatory state. In some embodiments, the subject has cancer, which is optionally a hematological malignancy or a solid tumor. In some embodiments, the T cell is a CAR-T cell.

In some embodiments, the cell population is a B lymphocyte population, and is capable of engraftment in a spleen or secondary lymphoid tissue of a subject. B-cell populations according to this disclosure have the potential to partially reconstitute humoral immunity in an immune compromised patient, for example, providing protection from or treatment for infectious diseases, including viral, bacterial, fungal, or parasite infection. In various embodiments, the B cells according to this disclosure are capable of differentiation to plasma cells for production of antigen-specific antibodies in vivo. In other embodiments, B cells produced according to this disclosure can be employed for cancer immunotherapy. In some embodiments, chimeric antigen B cells (CAR B cells) are prepared by gene modifications at iPSC, embryonic bodies, hCD34+ cells, hematopoietic progenitor cell, or B cell level. CAR B cells express a surface BCR and/or secrete a recombinant monoclonal antibody that recognizes a target antigen, such as a cancer antigen or an infectious disease antigen. In still other embodiments, B cells produced according to this disclosure are used for ex vivo production of antibodies (e.g., vaccine antibodies for providing protection from an infectious agent).

In some embodiments, the cell population is a monocyte or macrophage cell population, and the cell population is capable of engraftment and maturation in various tissues of a subject, including tumors. In various embodiments, the monocyte or macrophage cell population is able to form tissue resident macrophages in a subject. In various embodiments, the macrophages are predominately of the M1 (pro-inflammatory) or M2 (immunosuppressive) phenotype. In various embodiments, the subject in need to treatment has a cancer of any of various tissues or organs, liver or kidney inflammatory disease, or bacterial infection (e.g., sepsis or infection or colonization of an indwelling medical device).

In some embodiments, the cell population is a megakaryocyte population, or is platelets developed therefrom. These cells or platelets are useful for treating inherited platelet defects, impacting for example, coagulation pathways.

In some embodiments, the cell population is a red cell population.

In some embodiments, the cell populations (or platelets) are derived from autologous cells or universally compatible donor cells or HLA-modified or HLA null cells (e.g., as described herein). That is, the cell populations are generated from iPSCs that were prepared from cells of the recipient subject or prepared from donor cells (e.g., universal donor cells, HLA-matched cells, HLA-modified cells, or HLA-null cells).

In other aspects, the invention provides a method for cell therapy, comprising administering the cell population described herein, or pharmaceutically acceptable composition thereof, to a human subject in need thereof. In various embodiments, the methods described herein are used to treat blood (malignant and non-malignant), bone marrow, and immune diseases. In various embodiments, the human subject has a condition comprising one or more of lymphopenia, a cancer, an immune deficiency, an autoimmune disease, a skeletal dysplasia, hemoglobinopathies, an anemia, a bone marrow failure syndrome, and a genetic disorder (e.g., a genetic disorder impacting the immune system). In some embodiments, the subject has cancer, such as a hematological malignancy or a solid tumor.

In some embodiments, the subject has a condition selected from acute myeloid leukemia; acute lymphoblastic leukemia; chronic myeloid leukemia; chronic lymphocytic leukemia; myeloproliferative disorders; myelodysplastic syndromes; multiple myeloma; Non-Hodgkin lymphoma; Hodgkin disease; aplastic anemia; pure red-cell aplasia; paroxysmal nocturnal hemoglobinuria; Fanconi anemia; thalassemia major; sickle cell anemia; severe combined immunodeficiency (SCID); Wiskott-Aldrich syndrome; hemophagocytic lymphohistiocytosis; inborn errors of metabolism; severe congenital neutropenia; Shwachman-Diamond syndrome; Diamond-Blackfan anemia; and leukocyte adhesion deficiency.

Cell lineages generated using the methods described herein are administered to the subject e.g., by intravenous infusion. In some embodiments, the methods can be performed following myeloablative, non-myeloablative, or immunotoxin-based (e.g., anti-c-Kit, anti-CD45, etc.) conditioning regimes.

As used herein, the term “about” means±10% of the associated numerical value.

Certain aspects and embodiments of this disclosure are further described with reference to the following examples.

### EXAMPLES

### Example 1—ETV2 Over-Expression Increases the Yield of Hemogenic Endothelial Cells and Enhances the CD34⁺ Cell Formulation During iPSC Differentiation but does not Affect Pluripotency

### Methods

iPSCs were developed from hCD34+ cells by episomal reprogramming as known in the art and essentially as described in Yu, et al. Induced pluripotent stem cell lines derived from human somatic cells, Science 318, 1917-1920, (2007); and J. Yu, et al. Human induced pluripotent stem cells free of vector and transgene sequences. Science 324, 797-801, (2009). Embryoid Bodies and hemogenic endothelium differentiation was performed essentially as described in: R. Sugimura, et al., Haematopoietic stem and progenitor cells from human pluripotent stem cells. Nature 545, 432-438, (2017); C. M. Sturgeon, et al, Wnt signaling controls the specification of definitive and primitive hematopoiesis from human pluripotent stem cells. Nat Biotechnol 32, 554-561, (2014); J. Yu, et al. Induced pluripotent stem cell lines derived from human somatic cells. Science 318, 1917-1920, (2007); and J. Yu, et al. Human induced pluripotent stem cells free of vector and transgene sequences. Science 324, 797-801, (2009).

Briefly, hiPSC were dissociated and resuspended in media supplemented with L-glutamine, penicillin/streptomycin, ascorbic acid, human holo-Transferrin, monothioglycerol, BMP4, and Y-27632. Next, cells were seeded in 10 cm dishes (EZSPHERE or low attachment plate) for the EB formation. On Day 1, bFGF and BMP4 were added to the medium. On Day 2, the media was replaced with a media containing SB431542, CHIR99021, bFGF, and BMP4. On Day 4, the cell media was replaced with a media supplemented with VEGF and bFGF. On day 6, the cell media was replaced with a media supplemented with bFGF, VEGF, interleukin (IL)-6, IGF-1, IL-11, SCF, and EPO. Cells were maintained in a 5% CO₂, 5% O₂, and 95% humidity incubator. To harvest the CD34+ cells, the EBs were dissociated on day 8, cells were filtered through a 70 μm strainer, and CD34+ cells were isolated by CD34 magnetic bead staining.

### Results

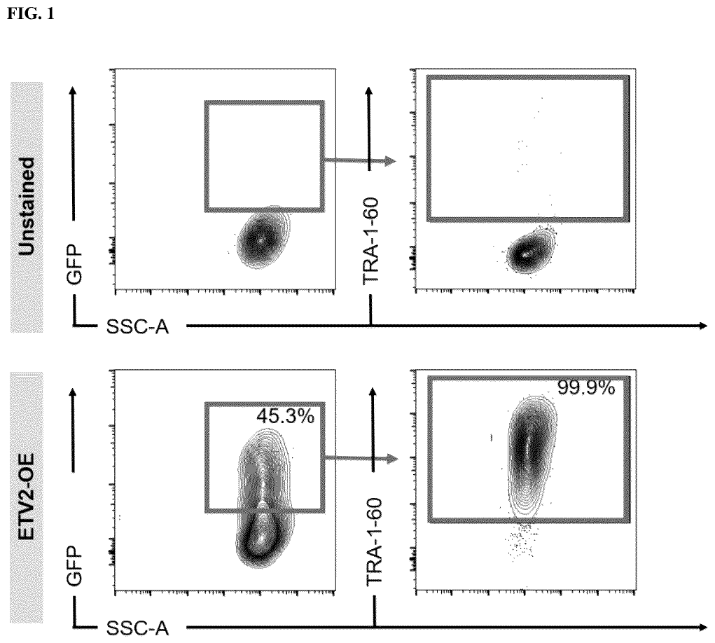

An adenoviral vector containing both ETV2 and GFP sequences under the control of the EF1A promoter was used to transduce induced pluripotent stem cells (iPSCs). After the transduction, about 45% of the iPSC culture was observed to be GFP positive, thus confirming ETV2 overexpression (ETV2-OE). It was further observed that ETV2-OE in iPSC cells preserves the pluripotency properties of iPSCs as shown by the stemness marker expression TRA-1-60 (FIG. 1). FIG. 1 shows FACS plots representative of transduction efficiency of iPSC with an adenoviral vector to overexpress the ETV2 and the GFP sequences.

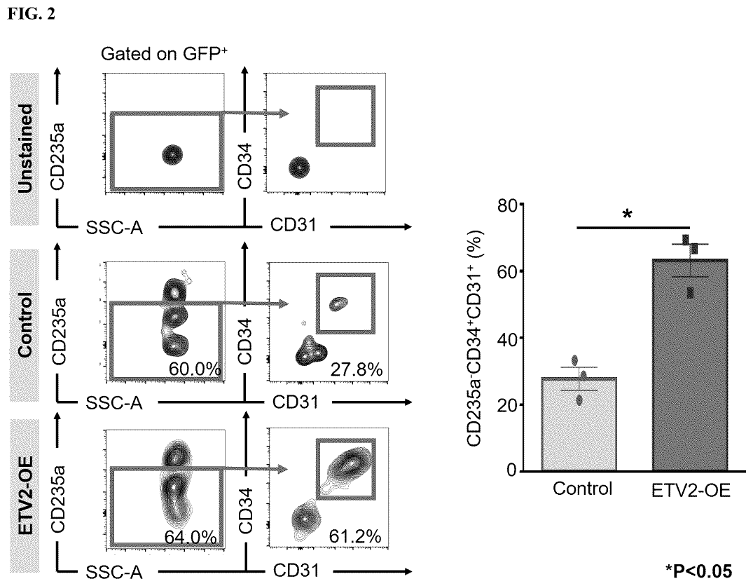

Next, the ETV2-OE-iPSCs were differentiated (along with control iPSCs transduced with a vector bearing the GFP sequence without ETV2) to embryoid bodies and subsequently to hemogenic endothelial cells (Strugeon et al., 2014). The results suggest that the overexpression of ETV2 boosts the formation of hemogenic endothelial cells as demonstrated by the expression of the CD34⁺ and CD31⁺ markers within the CD235a⁻ population (FIG. 2). Specifically, FIG. 2 shows representative flow cytometric analysis of hemogenic endothelial cells (defined here as CD235a−CD34+CD31+) and relative quantification demonstrates that ETV2-OE enhances the formation of hemogenic endothelial cells as compared to controls.

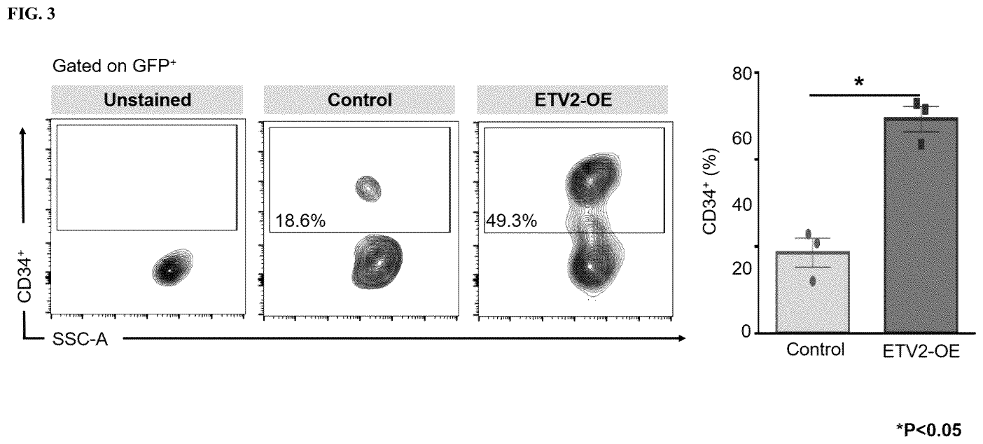

Moreover, the results suggest that ETV2-OE enhances the formation of the CD34⁺ cells (FIG. 3). FIG. 3 shows representative flow cytometric analysis of CD34+ cells and relative quantification demonstrates that ETV2-OE enhances the CD34+ cell formation.

Overall, these data indicate that ETV2 overexpression in iPSCs does not affect their pluripotency properties and facilitates their ability to undergo the hemogenic endothelial and hematopoietic differentiations.

### Example 2—iPSC-Derived HSCs Generated with EHT Undergo T Cell Differentiation Similar or Better than Bone Marrow-Derived HSCs

### Methods

To analyze the EHT, EB-derived CD34+ cells were suspended in medium containing Y-27632, TPO, IL-3, SCF, IL-6, IL-11, IGF-1, VEGF, bFGF, BMP4, and FLT3. After the cells had adhered to the bottom of the wells for approximately 4-18 hours (by visual inspection), Yoda1 was added to the cultures. After 4-7 days, the cells were collected for analysis.

iPSCs were differentiated to embryoid bodies for 8 days. At day 8, CD34+ cells from iPSC-derived embryoid bodies were harvested and cultured for additional 5 to 7 days to induce endothelial-to-hematopoietic (EHT) transition. Then, CD34+ cells were harvested from the EHT culture between day 5 to day 7 for further hematopoietic lineage differentiation.

CD34+ cells, harvested from the EHT culture between day 5-7 (or total of day 13-21 differentiation from iPSCs), were seeded in 48-well plates pre-coated with rhDL4 and RetroNectin. T lineage differentiation was induced in media containing aMEM, FBS, ITS-G, 2BME, ascorbic acid-2-phosphate, Glutamax, rhSCF, rhTPO, rhIL7, FLT3L, rhSDF-1a, and SB203580.

Between day 2 to day 6, 80% of the media was changed every other day. At D7, cells were transferred into new coated plates and analyzed for the presence of pro-T cells (CD34+CD7+CD5+/−).

Between day 8 to day 13, 80% of the media was changed every other day. At D14, 100,000 cells/wells were transferred to a new coated plate and the cells analyzed for the presence of pre-T cells (CD34− CD7+CD5+/−).

Between day 15 to day 20, 80% of the media was changed every other day. Cells were harvested at D21, and the cells were analyzed for CD3, CD8, CD5, CD7, TCRab expression, as surrogates for T cells, via FACS, and/or activated using CD3/CD28 beads to evaluate their functional properties.

After 21 days of differentiation, cells were collected and re-seeded at approximately 80,000 cells into new 96-well culture plates in RPMI 1640 (no L-glutamine; no phenol red) plus FBS, L-glutamine, IL-2, and then activated with 1:1 CD3/CD28 beads. After 72 hours of activation with CD3/CD28 beads, cells were analyzed for CD3, CD69, CD25 expression by FACS and IFN-γ expression using RT-qPCR. The supernatant was analyzed by ELISA.

### Results

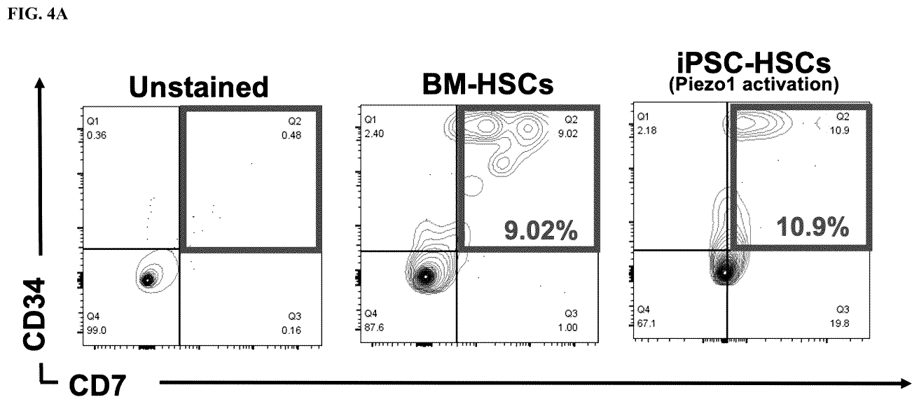

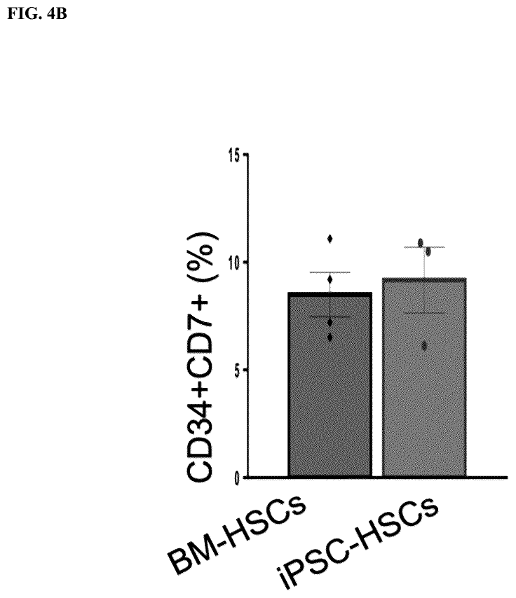

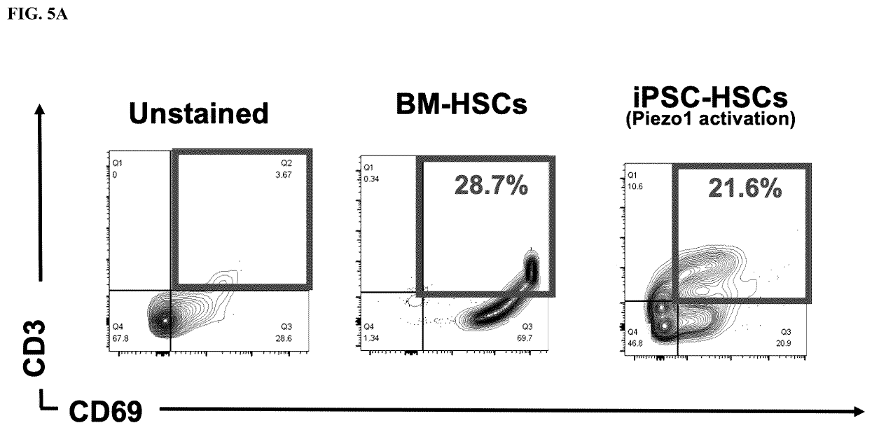

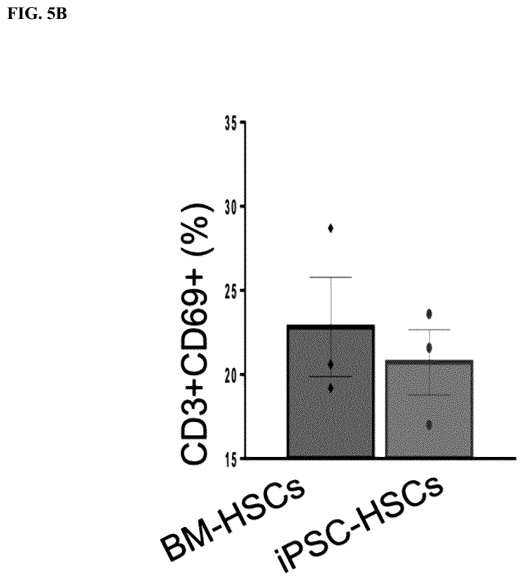

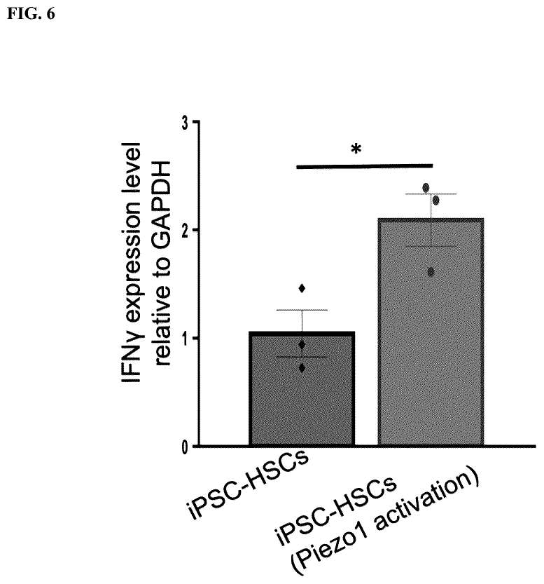

FIG. 4A and FIG. 4B show that iPSC-derived HSCs that are derived with Piezo1 activation undergo pro-T cell differentiation similar to bone marrow (BM)-HSCs. Further, FIG. 5A and FIG. 5B show that iPSC-derived HSCs generated with Piezol activation undergo T cell differentiation and can be activated with CD3/CD28 beads similar to BM-HSCs. FIG. 6 shows that iPSC-derived HSCs generated with Piezol activation can differentiate to functional T cells, as demonstrated by INFγ expression upon stimulation with CD3/CD28 beads. Together, these results demonstrate that Piezol activation during HSC formation enhances HSC ability to further differentiate to progenitor T cells and functional T cells ex vivo.

### REFERENCES

1. Nianias, A. & Themeli, M. Induced Pluripotent Stem Cell (iPSC)-Derived Lymphocytes for Adoptive Cell Immunotherapy: Recent Advances and Challenges. Curr Hematol Malig Rep 14, 261-268 (2019).
2. Brauer, P. M., Singh, J., Xhiku, S. & Zúñiga-Pflücker, J. C. T Cell Genesis: In Vitro Veritas Est? Trends Immunol 37, 889-901 (2016).
3. Kennedy, M. et al. T Lymphocyte Potential Marks the Emergence of Definitive Hematopoietic Progenitors in Human Pluripotent Stem Cell Differentiation Cultures. Cell Reports 2, 1722-1735 (2012).
4. Sturgeon, C. M., Ditadi, A., Awong, G., Kennedy, M. & Keller, G. Wnt Signaling Controls the Specification of Definitive and Primitive Hematopoiesis From Human Pluripotent Stem Cells. Nat Biotechnol 32, 554-561 (2014).
5. Chang, C.-W., Lai, Y.-S., Lamb, L. S. & Townes, T. M. Broad T-Cell Receptor Repertoire in T-Lymphocytes Derived from Human Induced Pluripotent Stem Cells. PLOS One 9, (2014).
6. Nishimura, T. et al. Generation of Rejuvenated Antigen-Specific T Cells by Reprogramming to Pluripotency and Redifferentiation. Cell Stem Cell 12, 114-126 (2013).
7. Themeli, M. et al. Generation of tumor-targeted human T lymphocytes from induced pluripotent stem cells for cancer therapy. Nat Biotechnol 31, 928-933 (2013).
8. Vizcardo, R. et al. Regeneration of Human Tumor Antigen-Specific T Cells from iPSCs Derived from Mature CD8+ T Cells. Cell Stem Cell 12, 31-36 (2013).
9. Montel-Hagen, A. et al. Organoid-induced differentiation of conventional T cells from human pluripotent stem cells. Cell Stem Cell 24, 376-389.e8 (2019).
10. Guo, R. et al. Guiding T lymphopoiesis from pluripotent stem cells by defined transcription factors. Cell Research 30, 21-33 (2020).
11. Nagano, S. et al. High Frequency Production of T Cell-Derived iPSC Clones Capable of Generating Potent Cytotoxic T Cells. Molecular Therapy—Methods & Clinical Development 16, 126-135 (2020).
12. Iriguchi, S. et al. A clinically applicable and scalable method to regenerate T-cells from iPSCs for off-the-shelf T-cell immunotherapy. Nature Communications 12, 430 (2021).

1. A method for preparing a cell population of a hematopoietic lineage, the method comprising:
preparing a pluripotent stem cell (PSC) population;
enriching for CD34+ cells to prepare a CD34-enriched population;
inducing endothelial-to-hematopoietic transition of the CD34-enriched population to prepare a population comprising hematopoietic stem cells (HSCs), and optionally harvesting cells from CD34-enriched population undergoing endothelial-to-hematopoietic transition; and
differentiating the HSC population to a hematopoietic lineage.

2. The method of claim 1, wherein the PSC population is a human iPSC population derived from lymphocytes, cord blood cells, peripheral blood mononuclear cells, CD34+ cells, or human primary tissues.

3. The method of claim 2, wherein the PSC population is derived from CD34-enriched cells isolated from peripheral blood.

4. The method of claim 2, wherein the iPSCs are homozygous for one or more HLA Class I and/or Class II genes.

5. The method of claim 2, wherein the iPSCs are gene-edited to delete one or more HLA Class I genes, delete one or more Class II genes, and/or delete one or more genes governing HLA or MHC expression or presentation capacity.

6. The method of claim 5, wherein the one or more genes governing HLA or MHC expression or presentation capacity is β2-microglobulin and/or CIITA.

7. The method of any one of claims 1 to 6, wherein CD34-enrichment and endothelial-to-hematopoietic transition is induced at Day 8 to Day 15 of iPSC differentiation.

8. The method of claim 7, wherein the endothelial-to-hematopoietic transition generates an HSC population comprising one or more of long-term hematopoietic stem cells (LT-HSCs), short-term hematopoietic stem cells, and hematopoietic stem progenitor cells.

9. The method of claim 7, wherein CD34+ cells are harvested from culture undergoing endothelial-to-hematopoietic transition, including harvesting of CD34+ floater and/or adherent cells.

10. The method of claim 8, wherein the HSC population comprises long-term hematopoietic stem cells (LT-HSCs)

11. The method of claim 7, where the induction of endothelial-to-hematopoietic transition comprises increasing the expression or activity of dnmt3b.

12. The method of claim 7, wherein the induction of endothelial-to-hematopoietic transition comprises applying cyclic stretch to the CD34-enriched cells.

13. The method of claim 12, wherein the cyclic stretch is 2D, 3D, or 4D cyclic stretch.

14. The method of claim 7, wherein the induction of endothelial-to-hematopoietic transition comprises Piezol activation.

15. The method of claim 14, wherein the Piezol activation is by contacting the CD34-enriched cells or fraction thereof with one or more Piezol agonists, which are optionally selected from Yoda1, Jedi1, Jedi2, or analogues or derivatives thereof.

16. The method of claim 7, wherein the induction of endothelial-to-hematopoietic transition comprises Trpv4 activation.

17. The method of claim 16, wherein the Trpv4 activation is by contacting the CD34-enriched cells with one or more Trpv4 agonists, which are optionally selected from GSK1016790A, 4alpha-PDD, or analogues or derivatives thereof.

18. The method of any one of claims 1 to 17, wherein the hematopoietic lineage is selected from progenitor-T cells, T lymphocytes, B lymphocytes, Natural Killer cells, neutrophils, monocyte, macrophages, red cells, megakaryocytes, and platelets.

19. The method of claim 18, wherein the HSC population or fraction thereof is differentiated ex vivo to progenitor T cells, T cells, NK cells, and/or fractions or analogous thereof.

20. The method of claim 19, wherein the HSC population or fraction thereof is cultured with a partial or full Notch ligand to produce a population comprising CD7+ progenitor T cells or a derivative cell population.

21. The method of claim 20, wherein the CD7+ progenitor T cells express CD1a.

22. The method of claim 21, wherein the CD7+ progenitor T cells do not express CD34 or express a diminished level of CD34 compared to the HSC population.

23. The method of any one of claims 20 to 22, wherein the CD7+ progenitor T cells express CD5.

24. The method of any one of claims 20 to 23, wherein the Notch ligand comprises at least one of DLL1, DLL4, SFIP3, or a functional portion thereof.

25. The method of any one of claims 20 to 24, wherein the Notch ligand is immobilized, functionalized, and/or embedded in 2D or 3D culture system.

26. The method of any one of claims 20 to 25, wherein the Notch ligand is incorporated along with a component of extracellular matrix, optionally selected from fibronectin, RetroNectin, and laminin, derivates or analogues thereof, and/or combinations thereof.

27. The method of claim 26, wherein the Notch ligand and/or component of extracellular matrix are embedded in inert materials providing 3D culture conditions, optionally selected from cellulose, alginate, and combinations thereof.

28. The method of any one of claim 26 or 27, wherein the Notch ligand, a component of extracellular matrix, or combinations thereof, are in contact with culture conditions providing topographical patterns and/or roughness to cells.

29. The method of any one of claims 20 to 28, wherein the Notch ligand, a component of extracellular matrix, topographical patterns and/or roughness, or combinations thereof, are cultured with cytokines and/or growth factors optionally selected from one or more of TNF-alpha and SHH.

30. The method of any one of claims 20 to 29, wherein the HSC population or fraction thereof is cultured in an artificial thymic organoid.

31. The method of any one of claims 20 to 30, comprising generating a T cell lineage from the progenitor T cells.

32. The method of claim 31, wherein the T cell lineage expresses at least one CD3 and a T cell receptor.

33. The method of claim 32, wherein the T cell lineage is CD8+ and/or CD4+.

34. The method of any claims from 31 to 33, wherein the T cell lineage expresses a chimeric antigen receptor (CAR).

35. The method of claim 31, wherein the T cell lineage is a regulatory T cell.

36. The method of claim 31, wherein the T cell lineage is a gamma-delta T cell.

37. The method of claim 31, wherein the T cell lineage is a alpha-beta T cell.

38. The method of claim 31, wherein the T cell lineage is a cytotoxic T cell.

39. The method of claim 31, comprising generating a natural killer (NK) cell population from the progenitor T cells.

40. The method of claim 39, wherein the NK cell lineage expresses a chimeric antigen receptor (CAR).

41. A T cell or NK cell population, or pharmaceutically-acceptance composition thereof, produced by the method of any one of claims 19 to 40.

42. The T cell or NK cell population of claim 41, wherein the cell population is capable of engraftment in a thymus or secondary lymphoid organ.

43. A method for cell therapy, comprising administering the T cell or NK cell population or pharmaceutically acceptable composition thereof of claim 41 or claim 42, to a human subject in need thereof.

44. The method of claim 43, wherein the human subject has a condition comprising one or more of lymphopenia, a cancer, an immune deficiency, an autoimmune disease, viral infection, a skeletal dysplasia, and a bone marrow failure syndrome.

45. The method of claim 44, wherein the subject has cancer, which is optionally a hematological malignancy or a solid tumor.

46. The method of claim 18, wherein the HSC population or fraction thereof is differentiated to B lymphocytes or derivatives thereof.

47. A B lymphocyte population or pharmaceutically acceptable composition thereof, produced by the method of claim 46.

48. The B lymphocyte population of claim 47, wherein the B lymphocyte population engrafts in the spleen or secondary lymphoid tissues of a subject.

49. A method for cell therapy or vaccination, comprising administering the B lymphocyte population, or pharmaceutically acceptable composition thereof of claim 47 or claim 48, to a human subject in need thereof.

50. The method of claim 49, wherein the human subject is immune compromised.

51. The method of claim 49 or 50, wherein the human subject is in need of antibody therapy or vaccination for immediate treatment and/or to gain protective immunity.

52. The method of claim 50 or claim 51, wherein the subject has or is at risk of viral, bacterial, fungal, or parasitic infection.

53. The method of claim 49 or claim 50, wherein the subject has or is at risk of cancer.

54. The method of any one of claims 49 to 53, wherein the B cell is a CAR-B cell.

55. The method of claim 18, wherein the HSC population or fraction thereof is differentiated to neutrophils, monocytes or macrophages.

56. A neutrophil, monocyte, or macrophage population or a pharmaceutically acceptable composition thereof, and produced by the method of claim 55.

57. A method for cell therapy, comprising administering the neutrophil, monocyte or macrophage population or pharmaceutically acceptable composition thereof of claim 56 to a human subject in need thereof.

58. The method of claim 57, wherein the subject has a condition selected from cancer, acquired or genetic hematological disease, liver or kidney inflammatory disease, or bacterial infection.

59. The method of claim 18, wherein the HSC population or fraction thereof is differentiated to megakaryocytes, and optionally to platelets.

60. A megakaryocyte population of platelet population derived therefrom or a pharmaceutically acceptable composition thereof produced by the method of claim 59.

61. A method for cell therapy, comprising administering the megakaryocyte or platelet population or pharmaceutically acceptable composition thereof of claim 60 to a human subject in need thereof.

62. The method of claim 61, wherein the subject has an inherited or acquired platelet defect.

63. The method of claim 18, wherein the HSC population or fraction thereof is differentiated to red cells or derivatives thereof.

64. A red cell population or a population derived therefrom or a pharmaceutically acceptable composition thereof produced by the method of claim 63.

65. A method for cell therapy, comprising administering the cell population or pharmaceutically acceptable composition thereof of claim 64 to a human subject in need thereof.

66. The method of claim 65, wherein the subject has an inherited or acquired red cell disorder, bone marrow failure disorder, high-altitude-related physiological or pathological condition, condition related to chemical or radiation exposure, or the subject is undergoing HSC transplant.

67. The method of claim 65, wherein the pharmaceutical acceptable composition is used to deliver or encapsulate one or more drugs or oxygen carriers.

68. A method comprising:
generating an HSC population comprising human long-term hematopoietic stem cells (LT-HSCs) from human pluripotent stem cells, wherein the HSC population is derived by endothelial-to-hematopoietic transition of CD34+ cells; and
culturing the HSC population or cells isolated therefrom with a partial or full Notch ligand and/or component of an extracellular matrix to produce a population comprising CD7+ progenitor T cells or a derivative cell population.

69. The method of claim 68, wherein the human pluripotent stem cells are induced pluripotent stem cells (iPSCs).

70. The method of claim 69, wherein the human pluripotent stem cells are derived from lymphocytes, cord blood cells, peripheral blood mononuclear cells, CD34⁺ cells, or human primary tissues.

71. The method of claim 70, wherein the iPSC population is derived from CD34-enriched cells isolated from peripheral blood.

72. The method of claim 70, wherein the iPSC population is derived from T lymphocytes isolated from peripheral blood.

73. The method of any one of claims 68 to 72, wherein the iPSCs are homozygous for one or more HLA Class I and/or Class II genes.

74. The method of claim 73, wherein the iPSCs are gene-edited to delete one or more HLA Class I genes, one or more Class II genes, and/or delete one or more genes governing HLA or MHC expression or presentation capacity.

75. The method of claim 74, wherein the one or more genes governing HLA or MHC expression or presentation capacity is B2-microglobulin and/or CIITA.

76. The method of any one of claims 68 to 75, wherein the endothelial-to-hematopoietic transition of the CD34+ cells is induced at Day 8 to Day 15 of iPSC differentiation.

77. The method of claim 76, wherein CD34+ cells are harvested from culture undergoing endothelial-to-hematopoietic transition, including harvesting of CD34+ floater and/or adherent cells.

78. The method of claim 76 or 77, wherein the endothelial-to-hematopoietic transition generates an HSC population comprising one or more of long-term hematopoietic stem cells (LT-HSCs), short-term hematopoietic stem cells, and hematopoietic stem progenitor cells.

79. The method of any one of claims 76 to 78, wherein generating the HSC population comprises increasing the expression or activity of dnmt3b in CD34+ cells.

80. The method of any one of claims 68 to 79, wherein generating the HSC population comprises applying cyclic stretch to CD34+ cells.

81. The method of claim 80, wherein one or more cell populations are subjected to 2D, 3D, or 4D cyclic stretch, and the cells subjected to cyclic stretch are optionally selected from one or more of iPSCs, CD34+ cells, endothelial cells (ECs), and hemogenic endothelial cells (HECs).

82. The method of any one of claims 68 to 78, wherein generating the HSC population comprises Piezol activation.

83. The method of claim 82, wherein the cells subjected to Piezol activation are selected from one or more of iPSCs, EBs, CD34+ cells, ECs, HECs, and HSCs.

84. The method of claim 82 or 83, wherein the Piezol activation is by contacting the cells with one or more Piezo 1 agonists, which are optionally selected from Yoda1, Jedi1, Jedi2, or analogues or derivatives thereof.

85. The method of any one of claims 68 to 78, wherein generating the HSC population comprises Trpv4 activation of CD34+ cells.

86. The method of claim 85, wherein the Trpv4 activation is by contacting the pluripotent stem cells or cells differentiated therefrom with one or more Trpv4 agonists, which are optionally selected from GSK1016790A, 4alpha-PDD, or analogues or derivatives thereof.

87. The method of any one of claims 68 to 86, wherein the HSC population is derived from day 8 to day 17 differentiation of human iPSCs.

88. The method of any one of claims 68 to 87, wherein the CD7⁺ progenitor T cells express CD1a.

89. The method of any one of claims 68 to 88, wherein the CD7⁺ progenitor T cells do not express CD34, or express a diminished level of CD34 compared to the HSC population.

90. The method of any one of claims 68 to 89, wherein the CD7⁺ progenitor T cells express CD5.

91. The method of any one of claims 68 to 90, wherein the Notch ligand comprises at least one of DLL1 and DLL4, or a functional portion thereof.

92. The method of claim 91, wherein the Notch ligand comprises a DLL1 amino acid sequence, or a functional portion thereof.

93. The method of claim 91, wherein the Notch ligand comprises a DLL4 amino acid sequence, or a functional portion thereof.

94. The method of any one of claims 91 to 93, wherein the Notch ligand is immobilized, functionalized, and/or embedded in 2D or 3D culture system.

95. The method of claim 94, wherein the Notch ligand is incorporated along with one or more components of extracellular matrix, optionally selected from fibronectin, retronectin, and laminin.

96. The method of claim 95, wherein the Notch ligand and/or component of extracellular matrix are embedded in inert materials providing 3D culture conditions, optionally selected from cellulose, alginate, and combinations thereof.

97. The method of any one of claim 95 or 96, wherein the Notch ligand, a component of extracellular matrix, or combinations thereof, are in contact with culture conditions providing topographical patterns and/or roughness to cells.

98. The method of any one of claims 94 to 97, wherein the Notch ligand, a component of extracellular matrix, topographical patterns and/or roughness, or combinations thereof, are cultured with cytokines and/or growth factors optionally selected from TNF-alpha, SHH, or combinations thereof.

99. The method of any one of claims 68 to 98, wherein the stem cell population is cultured in an artificial thymic organoid.

100. The method of any one of claims 68 to 99, comprising generating a derivative of the progenitor T cells or generating a T cell lineage from the progenitor T cells.

101. The method of claim 100, wherein the derivative of the progenitor T cell or T cell lineage express CD3 and a T cell receptor.

102. The method of claim 101, wherein the T cell lineage is CD8⁺ and/or CD4⁺.

103. The method of any one of claims 100 to 102, wherein the T cell lineage is modified to express a chimeric antigen receptor (CAR).

104. The method of claim 100, wherein the T cell lineage is a regulatory T cell.

105. The method of claim 100, wherein the T cell lineage is a gamma-delta T cell.

106. The method of claim 100, wherein the T cell lineage is a alpha-beta T cell.

107. The method of claim 100, wherein the T cell lineage is a cytotoxic T cell.

108. The method of claim 100, wherein the derivative of the progenitor T cell is a natural killer (NK) cell.

109. A CD7⁺ progenitor T cell or pharmaceutically acceptance composition thereof produced by the method of any one of claims to 68 to 99.

110. The CD7⁺ progenitor T cell or composition thereof of claim 109, wherein the progenitor T cell is CD34⁻ or CD34^{low}.

111. The CD7⁺ progenitor T cell of claim 109 or 110, wherein the progenitor T cell is capable of engraftment in a thymus or spleen upon administration.

112. A method for cell therapy, comprising administering the CD7⁺ progenitor T cell or pharmaceutically acceptable composition thereof of any one of claims 109 to 111 to a human subject in need of an increase in T cell numbers.

113. The method of claim 112, wherein the human subject has a condition comprising one or more of lymphopenia, a cancer, an immune deficiency, an autoimmune disease, a skeletal dysplasia, a bone marrow failure syndrome, and a viral infection.

114. The method of claim 113, wherein the subject has cancer.

115. The method of claim 114, wherein the cancer is a hematological malignancy.

116. The method of claim 114, wherein the subject has a solid tumor.

117. A derivative of the CD7⁺ progenitor T cell or T cell lineage produced by the method of any one of claims 100 to 108, or a pharmaceutically acceptable composition thereof.

118. The derivative of the CD7⁺ progenitor T cell of claim 117, wherein the derivative of the CD7⁺ progenitor cell or T cell lineage is capable of engraftment in a thymus or spleen.

119. A method for adoptive cell therapy, comprising administering the derivative of the CD7⁺ progenitor T cell or T cell lineage, or composition thereof, according to claim 117 or 118 to a human subject in need of an increase in T cell numbers.

120. The method of claim 119, wherein the human subject has a condition comprising one or more of a lymphopenia, a cancer, an immune deficiency, an autoimmune disease, a skeletal dysplasia, a bone marrow failure syndrome, and a viral infection.

121. The method of claim 120, wherein the subject has cancer.

122. The method of claim 121, wherein the cancer is a hematological malignancy.

123. The method of claim 121, wherein the subject has a solid tumor.
